(19) United States
(12) Patent Application Publication
(10) Pub. No.: US 2013/0303729 A1
(43) Pub. Date: Nov. 14, 2013

(54) Plasmon-assisted Pyrolysis for Site-specific Protein Digestion

(71) Applicants: Franco Basile, Fort Collins, CO (US); Rong Zhou, Laramie, WY (US)

(72) Inventors: Franco Basile, Fort Collins, CO (US); Rong Zhou, Laramie, WY (US)

(73) Assignee: Wyoming Research Products Center, Laramie, WY (US)

(21) Appl. No.: 13/495,819

(22) Filed: Jun. 13, 2012

Related U.S. Application Data
(63) Continuation-in-part of application No. 12/371,893, filed on Feb. 16, 2009.
Application No. 12/371,893 is a continuation-in-part of application No. 12/307,538, filed on Oct. 12, 2009.

(51) Int. Cl. C07K 1/12; B01J 19/12; B82Y 99/00

(52) U.S. Cl. 530/343; 530/407; 422/186; 977/777

(57) ABSTRACT

Apparatus using gold nano-particle surface Plasmon resonance heating for a rapid, reagentless and site specific cleavage at the C-terminal of aspartic acid and at the N-terminus of the amino acid cysteine in peptides and proteins induced by the thermal decomposition at 220-250° C. for 10 s in solid samples.

### BACKGROUND OF THE INVENTION

This invention relates generally to mass spectrometry characterization of peptides and proteins in the condensed phase and, more specifically, to apparatus using light and gold nanoparticles for the rapid, reagentless and site-specific decomposition and/or digestion of proteins and proteins in a sample for subsequent mass spectrometry analysis.

Pyrolysis (thermal lysis) has historically been used for the rapid preparation of samples for mass spectrometry (MS) analysis. Pyrolysis is defined as the decomposition reactions caused by thermal energy alone at temperatures above 300-350° C., while reactions caused by thermal energy at temperatures between 175-250° C. are considered thermal decompositions [1]. (Note: in previously published work from our laboratory, we have referred to all these processes as pyrolysis, for example, pyrolysis D-cleavage for the thermally induced cleavage at aspartic acid (D) [2]. However, in view of Moldoveanu's definitions of pyrolysis and thermal decomposition [1], we adopt in this manuscript the convention of referring to reactions conducted at temperatures between 150-300° C. as thermal decompositions, rather than pyrolysis). As a MS inlet, direct insertion probes (DIP) and/or pyrolysis have been used to analyze high molar mass samples by generating low molecular weight volatile molecules (i.e., thermal fragments) that are more amenable for analysis by electron ionization (EI) MS. Samples analyzed in this mode included high molecular weight synthetic polymers [3, 4], biomolecules [5, 6], and intact microorganisms [7-10]. Useful information regarding the monomer constituents in synthetic polymers could be obtained from mass spectra generated from these volatile thermal fragments. In the case of analysis of biological samples, the information yielded by these measurements was more limited due to the high complexity of the sample and the resulting mass spectra. Implementation of pattern recognition data analysis like principal components analysis (PCA) [11, 12] aided in the classification of complex mass spectral patterns with subtle differences between them. This strategy proved particularly successful for the classification of micro-organisms based on their mass spectral lipid profiles [13]. However, because DIP and/or Pyrolysis-MS was usually performed using EI, the measurement was limited to the analysis of volatile pyrolysis products of molar masses below about 1000 g/mol.

With the advent of electrospray ionization (ESI) [14-16] and matrix assisted laser desorption ionization (MALDI) [17-20], the analysis of high molar mass molecules was made possible and opened the possibility to analyze nonvolatile products of thermal decompositions. Latimer et al. [21, 22] first applied MALDI-MS for the analysis of nonvolatile pyrolysis products of synthetic polymers, detecting several species corresponding to the original polymer backbone. Meetani et al. later analyzed the nonvolatile thermal decomposition products (245° C.) of a series of poly-peptides with MALDI-MS, again detecting a series of high molecular mass products [23], the distribution of these products depending on the nature of the amino acids present in the peptide sequence. In a more recent study implementing LC-MS/MS, Meetani et al. also reported that thermal decomposition products of the leuenkephalin peptide resulted from a sequence ladder fragmentation through the formation of head-to-tail cyclic peptide fragments (and consequently with a −18 Da shift) [24]. Using a combination of MS and tandem MS (MS/MS) for the analysis of nonvolatile thermal decomposition products of peptides and proteins, our laboratory discovered that a site-specific fragmentation occurred at the C-terminus of aspartic acid (D) when peptides or proteins were subjected to temperatures between 200 and 250° C. for 10 s [2, 25]. The products of the thermal cleavage at D were easily identified by mass alone since these products resulted from a hydrolysis of the peptide bond C-terminus of D. Other products also detected corresponded to water and ammonia losses. Our study also demonstrated that peptides formed through the thermal decomposition of large proteins (e.g., lysozyme) preserved the sequence information of the original protein, and implementing a bottom-up proteomic approach (MS/MS measurement and database matching) a standard protein sample was identified from a peptide resulting from the thermal decomposition site-specific cleavage at D alone (using MASCOT MS/MS Ions Search) [2].

Recently, MALDI-MS imaging has been combined with bottom-up proteomics in order to identify proteins from intact tissue sections. Though the enzymatic digestion techniques can achieve site-specific cleavage, their application in MALDI-MS imaging (and in bottom-up proteomics in general) is limited to laboratory work by the narrow range of solution conditions for optimum activity of enzyme, long reaction times (>6 hours to overnight), stringent storage requirements (e.g., −20 to −80° C.), and requirements of dissolution or/and specific amino acids in samples. In addition, for MALDI-MS imaging protein identification, the trypsin solution has to be delivered within a narrow spatial range in order to avoid proteins and/or product peptide lateral diffusion. As a result, the trypsin solution must be delivered at specific spots (200 μm each, 250 μm apart) and in small volumes (15 nL), which requires repetitive solution deliveries within a spot to have sufficient enzyme for product (i.e., peptide) formation. For example, a tissue section of 1 cm×1 cm requires about 4 hours to be digested with trypsin, with each spot taking 30×15 nL enzyme solution depositions.

To overcome these disadvantages, especially those of long reaction times, our laboratory has developed a rapid and simple non-enzymatic method to digest proteins on tissue involving the thermal degradation of proteins. The method, as reported in our previous work, cleaves proteins and peptides at the C-terminal of aspartic acid (symbol D) and at the N-terminal of cysteine (symbol C) when the sample is subjected to temperatures between 220-250° C. for 10 s. The combined process is termed Thermal Degradation/Digestion or TDD. These initial studies were performed in a test-tube furnace pyrolyzer in order to induce site-specific cleavage on proteins and peptides in the condensed phase (i.e., solid samples).²⁻⁴

### SUMMARY OF THE INVENTION

The present invention includes the rapid, reagentless and site specific cleavage at the N-terminus of the amino acid cysteine (C) and C-terminus of aspartic acid (D) in peptides and proteins induced by the thermal decomposition at 220-250° C. for 10 s in solid samples. This thermally induced cleavage at C occurs under the same conditions and simultaneously to the previously reported thermally induced site-specific cleavage at the C-terminus of aspartic acid (D) (Zhang, S.; Basile, F. J. Proteome Res. 2007, 6, (5), 1700-1704). The C cleavage proceeds through cleavage of the nitrogen and α-carbon bond (N-terminus) of cysteine and produces modifications at the cleavage site with an amidation (−1 Da) of the N-terminal thermal decomposition product and a −32 Da mass change of the C-terminal thermal decomposition product, the latter yielding either an alanine or β-alanine residue at the N-terminus site. These modifications were confirmed by off-line thermal decomposition electrospray ionization (ESI)-MS, tandem MS (MS/MS) analyses and accurate mass measurements of standard peptides. Molecular oxygen was found to be required for the thermal decomposition and cleavage at C as it induced an initial cysteine thiol side chain oxidation to sulfinic acid. The cleavage at the C-terminus of D occurs through a hydrolysis of the peptide bond. Similar to the thermally induced D cleavage, missed cleavages at C were also observed. The combined thermally induced digestion process at D and C, termed thermal decomposition/digestion (TDD), was observed on several model proteins tested under ambient conditions and the site-specificity of the method confirmed by MS/MS.

Unlike the thermally induced cleavage at D, the thermally induced cleavage at C is accompanied by mass changes at the cleavage site of both the C- and N-terminal products. As a result of these mass changes (i.e., not resulting from a peptide bond hydrolysis), the cleavage at C remained undiscovered in previous work describing the cleavage at D [2]. Using a series of standard peptides and a ¹⁵N-stable isotope peptide, studies were performed with MS/MS and accurate mass measurements to confirm the cleavage site at C, the mass changes and nature of the observed products after the thermal cleavage under atmospheric conditions of peptides containing the amino acid C.

To extend the utility of the TDD technique, for example, to the sample preparation in MALDI-MS imaging for on-tissue protein identification, a photo-TDD method was developed using gold (Au) nanoparticles (NP) heat generation. In this method, Au-NP's of 50 nm in diameter interact with green light through a surface Plasmon resonance (SPR) process, which involves the resonant oscillation of Au free electrons with light. This SPR process in AU-NPs leads to heating of the Au-NP and subsequently its surroundings. Thus, by placing a layer of Au-NP's over the tissue sample and irradiating it with green light (wavelength of 532 nm) all sections exposed to light will digest, i.e., undergo TDD, into products in seconds. For example, proteins can be digested into peptides in 10 seconds. Other molecules may require different times to achieve thermal decomposition. This Photo-TDD process is only induced in the area that is exposed to light and has Au-NP. By adjusting laser intensity, coverage of Au-NP and/or exposure time, temperature around Au-NP can be controlled within the range of 220-250° C. so that site-specific cleavage from TDD can be induced on any protein sample, this regardless of its absorption maximum.

This Photo-TDD process with Au-NP's has been performed with a green laser and recently with a green Light Emitting Diode (LED) array. Because of the ability to focus laser down to small areas, this photo-TDD technique has the potential to perform spatially-resolved protein digestions. On the other hand, by using the LED array, large tissue areas can be “digested” within 10 s. As a result, the Photo-TDD technique can be applied in MALDI-MS imaging as a sample preparation method to generate peptide fragments by cleaving protein in appointed sites on a tissue section. That is, this approach can perform rapid and non-enzymatic on-tissue protein digestion for direct tissue (DT) protein identification.

### BRIEF DESCRIPTION OF THE FIGURES

FIGS. 1(a) and (b) are graphical representations of (a) the ESI-mass spectrum of the nonvolatile thermal decomposition (TD; 220° C., 10 s) products of the peptide SEQ ID NO: 1 PHCKRM, and (b) the tandem mass spectrum of the thermal decomposition product at m/z 505.5; ions labeled y* are the product of sequence permutation due to ion cyclization during the CID process (see text for details), while m/z values in bold in the fragmentation scheme denote that the ions were observed.

FIGS. 2(a)-(c) are graphical representations of (a) the ESI-mass spectrum of the nonvolatile thermal decomposition (TD; 220° C., 10 s) products of the peptide somatostatin (sequence SEQ ID NO: 2 AGCKNFFWKTFTSC), (b) the tandem mass spectrum of the thermal decomposition product at m/z 1535.6, and (c) the tandem mass spectrum of the thermal decomposition product at m/z 1375.6.

FIGS. 3(a) and (b) are graphical representations of the ESI-mass spectra of the nonvolatile thermal decomposition products for the peptide SEQ ID NO: 3 AWRGCLLFK (a) unlabeled and (b) ¹⁵N—C_{→} cysteine labeled.

FIGS. 4(a)-(c) are graphical representations of the tandem mass spectra of the AWRG nonvolatile thermal decomposition product of the peptide SEQ NO: 3 AWRGCLLFK (a) unlabeled (m/z 488), (b) an amidated standard peptide SEQ ID NO: 4 (AWRG-NH₂), and (c) ¹⁵N-labeled (m/z 489).

FIG. 5 is a graphical representation of the thermal decomposition (TD) cleavage at C product ratio under different atmospheres; the product ratio was calculated by dividing the peak intensity for the nonvolatile thermal decomposition product peptide SEQ ID NO: 14 (⁻³²CKRM, at m/z 505) to the intact peptide SEQ ID NO: 1 (PHCKRM), at m/z 771); error bars represent ±1 standard deviation, n=5.

FIGS. 6(a)-(c) are graphical representations of (a) the MALDI-mass spectrum of the thermal decomposition products of lysozyme at 220° C. (MALDI matrix: CHCA; reflectron mode), (b) the MALDI-mass spectrum of the thermal decomposition products of α-lactalbumin at 220° C. (MALDI matrix: CHCA; reflectron mode), and (c) the ESI-mass spectrum of the thermal decomposition products (220° C.) of α-lactalbumin; an asterisk indicates amidated C-terminus (ESI solvent: 1:1 methanol/0.1% aqueous formic acid; ESI voltage: 4 kV).

FIG. 7(a) illustrates the detailed diagram of an oscillating capillary nebulizer (OCN) used to spray samples, Au-NP and MALDI matrix solutions; FIG. 7(b) shows the setup of Photo-TDD. Laser used in the photo-TDD experiments was 150 mW of power and 532 nm of wavelength, and this laser was focused by lens with focal distance 38 mm and magnification 15 times; and FIG. 7(c) shows an LED array for exposing the entire sample area to light.

FIG. 8 shows the processes of photo-TDD experiment using a laser and Au-NP's.

FIG. 9 shows the spatial resolution of the AU-NP Photo-TDD process; the pyrolysis laser (aka Photo-TDD laser) is moved in the y-direction and the MALDI-laser was moved in the x-direction.

FIG. 10(a) illustrates the mass spectra of the peptide Angiotensin II before (inset) and after photo-TDD; FIG. 10(b) shows the mass spectra of the peptide antioxidant peptide-A before and after photo-TDD.

FIG. 11(a) shows results the photo-TDD experiment for another peptide, VIP (1-12), which contains two aspartic acid (D) residues; FIG. 11(b) shows the analysis by MALDI-MS of another peptide Bax, BH3, containing one cysteine residue and two aspartic acid residues, before and after photo-TDD.

### DETAILED DESCRIPTION OF PREFERRED EMBODIMENTS

The present invention includes the use of a localized source of heat to decompose and/or digest peptides and proteins. In a preferred embodiment, metal nanoparticles that generate heat upon exposure to light are used, in particular gold nanoparticles in the range of 30 nm to 90 nm that generate heat in response to exposure to a light source that includes green light preferentially about 532 nm, but other known localized sources of heat could be used as well. The gold nanoparticles are in a single layer, as demonstrated by scanning electron microscope imaging, on a sample of peptides and/or proteins to by analyzed. The sample preferably, but not necessarily, is on a flat substrate, such as a glass slide, and in the examples was applied at approximately 5 μg/cm². Decomposition and digestion of the peptides and proteins occurs between 220 and 250° C. Sufficient light intensity in the appropriate wavelength range is needed to heat the nanoparticles to the desired temperature range within the desired amount of time and can be adjusted appropriately to the conditions. In the examples, a 45.7 mW laser was used, supplying 203 W/cm², and it was found that the product yield was not dramatically affected across a light exposure range of between 2 and 50 seconds. Considering conservation of time and the introduction of instabilities in the experimental apparatus if too short of a cycle time is used, 10 seconds was used in the examples.

### EXAMPLE 1

### Experimental

### Chemicals

Peptides used were: (1) PHCₒₓKRM, where Cₒₓ is the C side chain oxidized to a sulfonic acid; (2) antioxidant peptide A, sequence SEQ ID NO: 1 PHCKRM; (3) somatostatin14, sequence SEQ ID NO: 2 AGCKNFFWKTFTSC; (4) AWRG(¹⁵N)CLLFK all from AnaSpec (San Jose, Calif.). The peptide AWRG-NH2 was purchased from American Peptide Co. (Sunnyvale, Calif.). The peptide of sequence SEQ ID NO: 4 AWRGCLLFK was synthesized using standard fluorenylmethyloxycarbonyl (FMOC) solid-phase synthesis on a PS-3 automated peptide synthesizer (Proteins Technologies, Inc.). The synthetic peptide was purified by reverse-phase HPLC and sample purity was verified by MALDI-MS. The proteins α-lactalbumin (bovine milk, FW 14.2 kDa) and lysozyme (chicken egg white, FW 14.3 kDa) were all from Sigma (St. Louis, Mo.) and used without further purification. The MALDI matrix α-cyano-4-hydroxycinnamic acid (CHCA) was from Sigma and used without further purification. All solvents [water, methanol, acetonitrile (ACN)] used for sample preparation and MS measurements were HPLC grade (Burdick and Jackson, Muskegon, Mich.), trifluoroacetic acid (TFA; Pierce Chemical Company, Rockford, Ill.), and the formic acid (FA 96%) were ACS reagent grade (Aldrich, St. Louis, Mo.).

### Pyrolyzer Design and Thermal Decomposition/Digestion (TDD) Procedure of Peptides and Proteins

Thermal Decomposition/Digestion (TDD) of peptides and proteins were conducted using a home-built pyrolyzer device [2]. Briefly, the pyrolyzer consisted of a glass tube (length 31 mm and internal diameter 4 mm; Agilent, Santa Clara, Calif.; part no. 5180-0841) and a resistance heating wire (Omega, Stamford, Conn., Nickel-Chromium wire; part no. NI60-015-50, length 20 cm) enwound around the tube. The pyrolyzer was heated by powering the resistance heating wire with alternating current (AC) from a transformer (model no. 3PN116C; Superior Electric, Farmington, Conn.). Temperature was measured in situ using a thermocouple probe (model HH12A; Omega Company, Stamford, Conn.) reaching down to the bottom of the glass tube. Approximately a 1 mg solid sample of the peptide or protein was placed in the pyrolyzer tube and was heated for 10 s under ambient conditions to a final temperature of 220° C. This corresponded to an AC voltage of approximately 13 V; however, final temperature (and as a result, applied voltage) depends highly on the pyrolyzer design and, thus, on the heat capacity of the pyrolyzer device. The heating time was controlled by an electronic digital timer (Gra-lab, model 655; Centerville, Ohio). After heating, the TDD nonvolatile residue was collected by washing/extracting the inside of the tube with several fractions totaling 1 mL of 50/50 (vol/vol) methanol/0.1% formic acid (FA) aqueous solution. This solution was used directly for ESIMS and/or MALDI-MS analyses.

The TDD process was also performed, in addition to laboratory atmospheric conditions (i.e., air), under different controlled atmospheres using N₂, NH₃, O2, or O₂+NH₃ gasses. Controlled atmospheric thermal decomposition experiments were performed with the furnace pyrolyzer described above enclosed inside a 20 mL glass vial, the latter fitted with a septum stopper and side holes for connections to the heating wire. The atmosphere inside the glass vial was flushed with the corresponding gas for about 2 min before heating. About 0.2 mg of the peptide antioxidant A (SEQ ID NO: 1 PHCKRM) was used for all of the controlled atmosphere experiments. The tube was heated to a maximum temperature of 220° C. (the applied voltage was adjusted to achieve the desired maximum temperature in order to account for the gasses different thermal conductivities). A total of five replicate samples were performed for each gas.

### Mass Spectrometry

The extracted solution of the TDD nonvolatile products were analyzed by direct infusion into a quadrupole ion-trap MS (LCQ classic, Thermo Finnigan, San Jose, Calif.) equipped with a micro-electrospray ionization (ESI) source. The sample was infused into the mass spectrometer at a flow rate of 3 μL/min via a 250-μL syringe (Hamilton, Holliston, Mass.) using the built-in LCQ syringe pump. The mass spectra were collected using the LCQ™ Tune Plus software (Thermo Finnigan, San Jose, Calif.). TandemMS (MS/MS) using collision induced dissociation (CID) was conducted with the following parameters: activation q of 0.250; isolation width was 1 Da and the percentage relative collision energy was in the range of 25%-40%, and was adjusted such that the relative abundance of the precursor ion in the product ion spectrum was approximately 30%-50% relative intensity.

MALDI-MS experiments were performed using either a Voyager DE-PRO or DE-STR (Applied Biosystems, Foster City, Calif.) instrument equipped with a N2 laser and operated in the reflectron mode. The matrix α-cyano-4-hydroxycinnamic acid (CHCA) was used for all measurements and was prepared by dissolving 10 mg of CHCA in a 1 mL solution of 1:1 acetonitrile/0.1% TFA aqueous solution. The solution containing the extracted TDD products was directly mixed with the matrix at different volume ratios, deposited (approximately 0.2 μL) and air-dried onto a MALDI plate. All MALDI-mass spectra were internally calibrated with either the intact peptide signal and/or a known TDD product peptide after its sequence was confirmed by ESI-MS/MS.

Accurate mass data were acquired using a hybrid linear ion trap/7-T Fourier transform (FT)-ion cyclotron resonance (ICR) MS (LTQ-FT; Thermo Electron, Bremen, Germany) equipped with a micro spray ion source (University of Utah Mass Spectrometry and Proteomics Core Facility). The ESI voltage, capillary voltage, capillary temperature and tube lens were set at 2.8 kV, 47 V, 175° C., and 150 V, respectively. The sheath gas (N₂) pressure was 50 psi with an auxiliary gas (N2) flow of 10 units. Peptides were analyzed in the positive ion mode. Peptides were dissolved in a solvent mixture of 50% ACN/0.1% aqueous FA and infused into the instrument at 3 μL/min flow rate. The FTMS was operated with a 50,000 resolution in the ICR cell. Accurate mass measurements were acquired using peptide internal standards. The peptides SEQ ID NO: 5 MRFA, SEQ ID NO: 6 FGFG, Angiotensin-III, SEQ ID NO: 7 YGGFM, SEQ ID NO: 8 YGGFLK, and SEQ ID NO: 9 YGGFL were used as internal standards. Internal standards were purchased from Sigma-Aldrich and used without further purification.

### Results and Discussion

Several peptide and protein standards were thermally decomposed at temperatures ranging between 220-240° C. and the nonvolatile products analyzed by a combination of ESI-MS, ESI-MS/MS, and MALDI-MS. Investigations were first carried out on low molar mass peptides in order to simplify interpretation of the resulting mass spectra, followed by a study involving high molar mass protein standards. The terminology used to describe the thermal degradation fragments is illustrated in Scheme 1, where N-terminal and C-terminal TDD products refer to the (neutral) product that retains either the N or C-terminus of the precursor peptide, respectively.

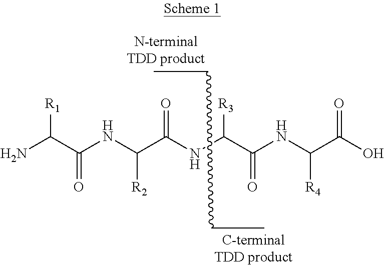

In order to implement the TDD approach as a protein digestion technique in proteomics, accurate knowledge of the chemical composition of the cleavage products is required. Because the thermal decomposition cleavage at C involves mass changes of both C- and N-terminal cleavage products, accurate mass measurements were performed on the C-terminal TDD product. Moreover, the TDD products of a stable-isotope-labeled peptide were analyzed by MS and MS/MS in order to elucidate the most likely structure of the products and the mechanism of fragmentation.

### Thermal Decomposition/Digestion (TDD) of Peptides

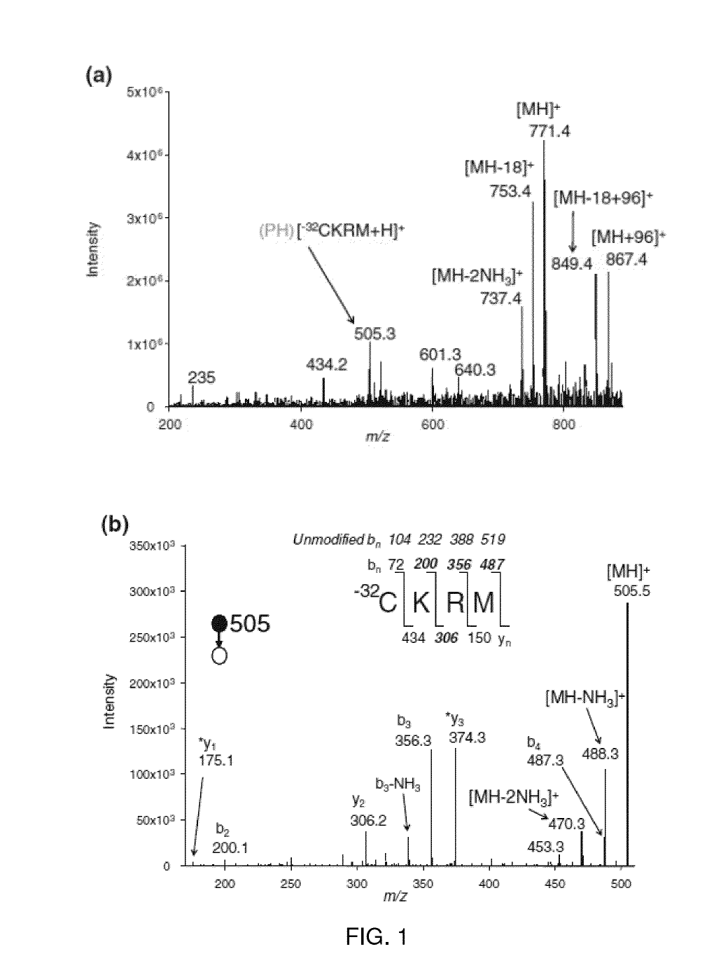

The peptide with the amino acid SEQ ID NO: 1 PHCKRM was heated under atmospheric conditions at 220° C. for 10 s using the tube furnace pyrolyzer. The nonvolatile thermal decomposition products were extracted and analyzed by direct infusion ESI-MS and the resulting mass spectrum is shown in FIG. 1a. The intact peptide protonated molecule, [MH]⁺, is observed at m/z 771.4, as well as ions corresponding to the loss of water (m/z 753.4) and consecutive losses of ammonias (m/z 737.4). The ion at m/z 505.5 is attributed to the peptide SEQ ID NO: 10 ⁻³²CKRM resulting from the cleavage at the N-terminus of C with an observed mass change of −32 Da (denoted by the left superscript −32 in C, that is, ⁻³²C in the sequence). This mass change is relative to the expected m/z 537.26 (not observed) for the [MH]⁺ ion of the peptide with the same amino acid sequence that would result from a hydrolysis cleavage of the peptide bond N-terminus to C. The signal corresponding to the [MH+96]⁺ ion at m/z 867.4 corresponds to the substitution of the C-terminus hydroxyl group with a trifluoroacetate group during the heating process (residual trifluoroacetic acid is present in most of these peptide samples from the LC purification step). To confirm the mass shift and sequence assignment of the ion at m/z 505.5, tandem MS (MS/MS) was performed and the resulting tandem mass spectrum is shown in FIG. 1b. Signals corresponding to b-ions (b₂, b₃, and b₄) and a y-ion (y₂) of the proposed sequence (and modification) were observed, as well as ions corresponding to losses of ammonia and water. From this tandem mass spectrum it can be noted that signals corresponding to b-ions are shifted by −32 Da (from the unmodified fragment ion), indicating that the assumed modification most likely occurs at the N-terminus of the peptide. Also observed were ions corresponding to internal scrambled sequence at m/z 175.1 (*y₁) and 374.3 (*y₃), due to the known cyclization of the precursor ion during the CID process [26-30], and correspond to the permutated sequence SEQ ID NO: 11 M⁻³²CKR, which includes the modified C residue.

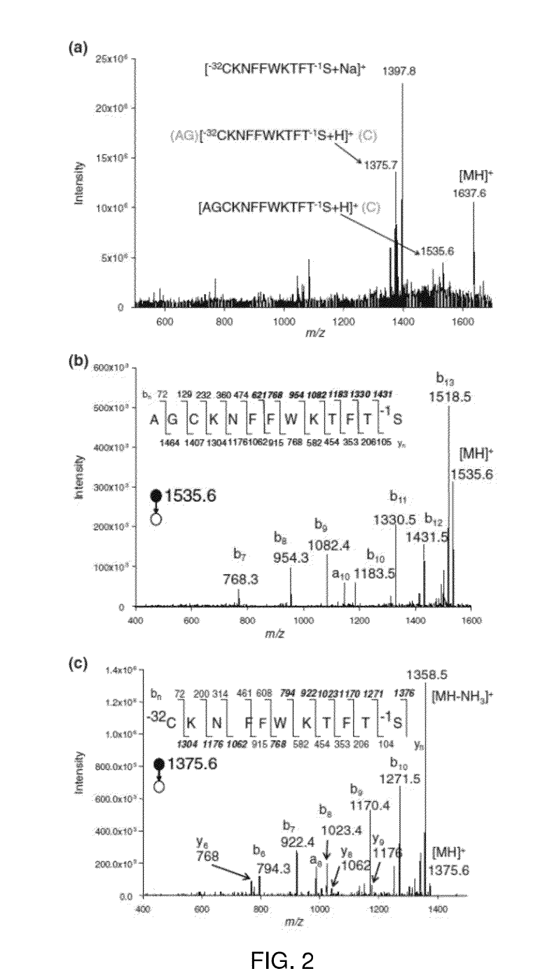

To gather further evidence for the thermal decomposition cleavage at C and possible modification(s) to the N-terminal thermal decomposition product (not observed in FIG. 1a), the peptide somatostatin (SEQ ID NO: 2 AGCKNFFWKTFTSC) was thermally degraded under similar conditions and analyzed by ESI-MS. This peptide contains two C in its sequence (C-3 and C-14), providing more opportunities to observe the mass changes produced by the thermal decomposition process. The ESI-mass spectrum of the nonvolatile thermal decomposition products is shown in FIG. 2a. The product due to thermal cleavage at the N-terminus of both C sites (C-3 and C-14) was observed at m/z 1375.7 (and its sodiated ion at m/z 1397.8) as well as the missed cleavage product (missed cleavage at C-3) at m/z 1535.6. These products illustrate the mass changes induced by the thermal decomposition process at the N- and C-termini of the C cleavage site: −32 Da at the C-terminus of C and −1 Da at the N-terminus of C (the latter indicated by the left superscript of −1 on the amino acid, that is, ⁻¹X, where X is any amino acid N-terminus to the C in the parent peptide). These combined mass changes are best illustrated with the thermal decomposition product at m/z 1375.7, where C-3 is modified by −32 Da and S-13 (N-terminus to C-14) is modified by −1 Da. Sequence confirmation of the products at m/z 1535.6 and 1375.6 was performed by MS/MS analysis and the results are shown in FIGS. 2b and c, respectively. In FIG. 2b the tandem mass spectrum of the ion at m/z 1535.6 shows signals corresponding to b-ions, b7 through b13, of the proposed sequence, which account for the −1 Da mass modification at the N-terminus of the peptide. Particularly prominent in this product ion mass spectrum is the ion at m/z 1518.5 corresponding to either NH₃ loss from the C-terminus amide or NH₃ loss from a lysine side chain. Also observed in this product ion mass spectrum are minor peaks corresponding to further NH₃ losses from the ions b₁₂ and b₁₁. Since these ions already have lost the C-terminus amino acids, it is most likely that these NH₃ losses are from the K side group. On the other hand, the NH₃ loss to form the b13 ion at m/z 1518.5 is most likely due to a NH₃ loss from a C-terminus amide. However, no y-ion series were observed in the product ion mass spectrum of this peptide to further confirm this rationalization. In FIG. 2c the tandem mass spectrum of the ion at m/z 1375.6 (SEQ ID NO: 12 ⁻³²CNFFWKTFT⁻¹S) shows signals corresponding to both y-ion and b-ion series, which also take into account mass modifications at both thermally induced cleavage sites. All detected signals corresponding to b-ions are shifted by −32 Da (from an unmodified fragment), again indicating that the observed chemical modification most likely occurs towards the N-terminus of the peptide. Likewise, signals corresponding to y-ions are shifted by −1 Da and it is in agreement with a modification at the C-terminus of the product peptide and the proposed sequence. Combined, these results confirm that the thermal decomposition cleavage at C occurs at its N-terminus and with chemical modifications at both the C-terminus of the product peptide and on the N-terminus of the cysteine-containing peptide. Also, these data point to the most likely formation of an amide on the C-terminus of the product peptide. However, no conclusions can be drawn from these results as to where the cleavage is taking place, that is, at the peptide bond (carbonyl carbon-nitrogen bond, CO—N) or at the nitrogen-α carbon bond (N—C_{α}) N-terminus to C. Experiments incorporating stable isotopes and accurate mass measurements were conducted to elucidate the most likely structure of the products and cleavage mechanism for the thermal decomposition cleavage at C.

### Characterization of the Thermal Decomposition N-Terminal Product of Peptides

The −1 Da mass change in the N-terminal peptide product can be attributed to: (1) C-terminus amide formation, and (2) allysine (aminoadipic semialdehyde) from the oxidation of lysine [31]. Even though amidation is the most common modification resulting in a −1 Da mass change, modification of lysine during the heating process cannot be ruled out since K is present in somatostatin (FIG. 2) and the same m/z values of the b-ions detected could also be attributed to the presence of allysine in the sequence. In order to gather conclusive evidence about the nature of the −1 Da mass shift in the N-terminus fragment to C and the nature of the fragmentation, a peptide with sequence SEQ ID NO: 3 AWRGCLLFK incorporating a nitrogen-15 (¹⁵N) stable isotope adjacent to the α-carbon (¹⁵N—C_{α}, Scheme 2) of the amino acid C was tested in a similar fashion.

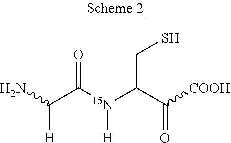 [SMILES: NCC(=O)[15NH]C(CS)C(=O)C(=O)O]

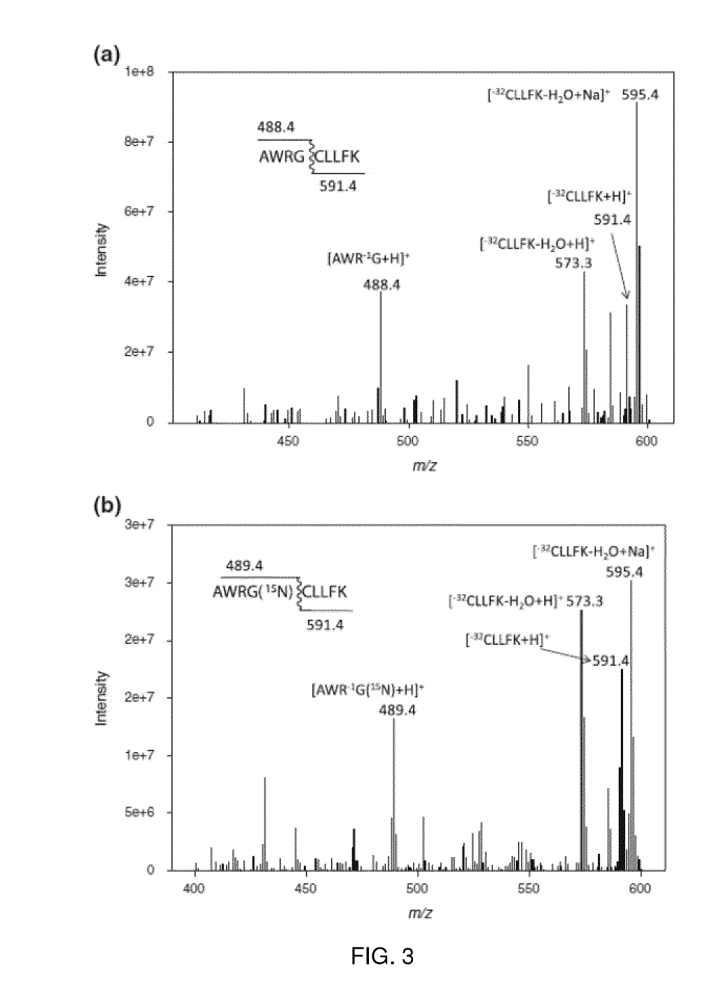

Using this peptide, observation of a +1 Da shift in the m/z value of either the N-terminal or C-terminal thermal decomposition product would indicate retention of the ¹⁵N adjacent to the α-carbon in C. Moreover, retention of the ¹⁵N by the N-terminus thermal decomposition product would provide evidence for the formation of an amide. The corresponding peptide containing an unlabeled nitrogen atom at C was also tested for comparison. FIG. 3 shows the ESI-mass spectra of the nonvolatile thermal decomposition products for the unlabeled (FIG. 3a) and ¹⁵N—C_{α} C labeled (FIG. 3b) peptides. The ion at m/z 488.4 in FIG. 3a can be assigned to the N-terminal thermal decomposition cleavage product of sequence AWR ¹G, where the C-terminus G has a −1 Da mass change. For the ¹⁵N-labeled peptide, the ESI-mass spectrum of the thermal decomposition products is shown in FIG. 3b and shows an ion at m/z 489.4, which corresponds to a +1 Da shift from the unlabeled peptide shown in FIG. 3a, with no other m/z values shifted by +1 observed. Consequently, results from these data can be rationalized by the formation of a C-terminus amide in the N-terminal thermal decomposition product by cleavage of the ¹⁵N—C_{α} bond of C, which incorporates the ¹⁵N atom into the N-terminal thermal decomposition product.

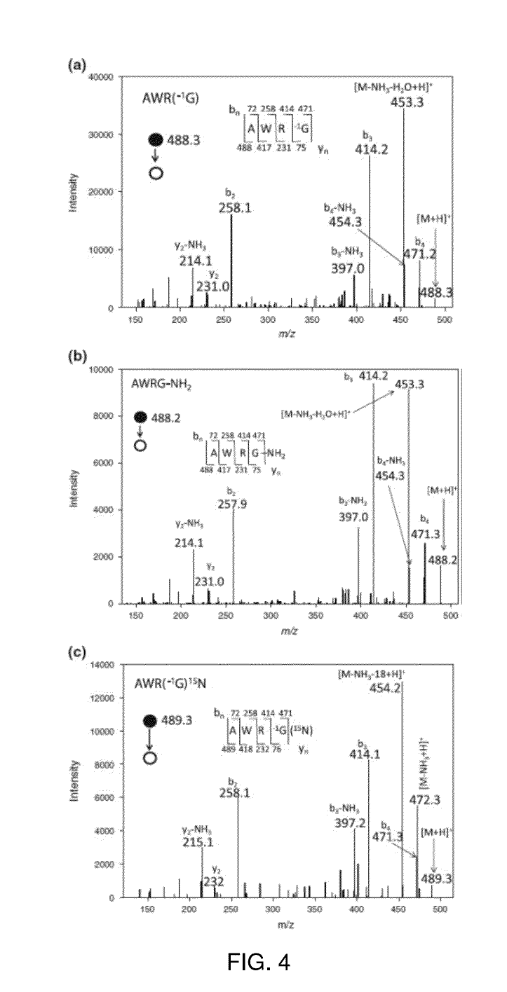

Further confirmation about the nature of the TD Nterminal product was gathered through MS/MS analyses of the unlabeled and ¹⁵N-labeled thermal decomposition products, and of an amidated standard peptide of the same sequence, and their product ion mass spectra (of ions at m/z 488 and 489) are shown in FIGS. 4a, b, and c, respectively. A quick survey of these product ion mass spectra shows that the fragmentation pattern and relative peak intensities are similar for these peptides; however, with several notable ions shifted by +1 Da in the ¹⁵N-labeled peptide in FIG. 4c. For example, the ions at m/z 471.1, 453.3, 231.0, and 214.1 in the non-labeled thermal decomposition product peptide (FIG. 4a) are observed shifted by +1 Da in the ¹⁵N-labeled peptide product ion mass spectrum (ions at m/z 472.3, 454.2, 232.0, and 215.1, respectively, in FIG. 4c). The ion at m/z 471 in FIG. 4a corresponds to the loss of −17 Da, or a loss of ammonia (NH₃), and in this case this loss can result from the C-terminus amide or from the arginine (R) side chain [32]. For an amide peptide, the b₄ ion would involve the loss of NH3 from the C-terminus and this ion would be of the same m/z value (m/z 471) for both the unlabeled and labeled peptides. FIG. 4c shows a strong signal at m/z 472 for the ¹⁵N-labeled peptide, indicating that the loss of NH₃ (−17 Da) from the R side chain is favored over the loss of ¹⁵NH₃ (−18 Da) from the C-terminus amide. In FIG. 4a a small contribution from the loss of −18 Da is also observed at m/z 470 and it is most likely attributed to a previously observed dehydration product during the CID of peptides involving peptide backbone oxygens [32, 33]. Also shifted by +1 Da in the product ion mass spectrum of ¹⁵N-labeled peptide is the ion at m/z 454.2. This ion can be rationalized by consecutive losses of NH₃ (side chain R) and H2O (backbone oxygen) and/or C-terminus ¹⁵NH₃. In the unlabeled peptide product ion mass spectrum (FIG. 4a) this consecutive losses yields an ion at m/z 453.2. A small contribution from the consecutive losses of R side chain NH₃ and C-terminus NH₃ is also observed at m/z 454 in FIG. 4a. This last observation indicates that the observed −18 Da loss in the labeled peptide (FIG. 4c) is most likely due to dehydration via backbone oxygen [33], with a small contribution from the loss of ¹⁵NH₃ from the C-terminus. Key indicators of the presence of an amidated peptide resulting from the thermal decomposition cleavage at the N-terminus of C are the fragment ions y₂ and y₂-NH₃ in FIGS. 4a and c. The y₂ ion in the unlabeled peptide product ion mass spectrum is detected at m/z 231 (FIG. 4a), while in the labeled peptide product ion mass spectrum is detected at m/z 232. In addition to these ions, the y₂-NH₃ ions resulting from the loss of NH3 from the R side chain in both native and labeled peptides and are observed at m/z 214.1 and 215.1, respectively. Finally, the thermal decomposition product (FIG. 4a) and the standard amidated peptide (FIG. 4b) product ion mass spectra are very similar, with all major peaks matching in m/z value and intensity. Collectively, these data are consistent with the formation of an amide peptide resulting from the cleavage of the N—C_{α} bond on the N-terminus of C during the thermal decomposition process.

### Characterization of the C-Terminal Product of the Thermal Decomposition Cleavage at Cysteine

The resulting C-terminal product peptide of the thermal decomposition cleavage at C is characterized by a −32 mass change when compared with a peptide of the same sequence without any modifications. For example, a peptide of the sequence SEQ ID NO: 13 CLLFK would produce a [M+H]⁺ at m/z 623.5, while the observed [M+H]⁺ of the C-terminal thermal decomposition product, supposedly comprised of the same amino acid sequence, is at m/z 591.5. Evidence acquired via MS/MS measurements of these C-terminal products (FIGS. 1b and 2c) point to an N-terminus modification of the peptide with a net mass change of −32 Da. It is also known from our stable isotope studies using ¹⁵N that the nitrogen next to the α-carbon is not incorporated into any of the ions corresponding to the C-terminal thermal decomposition products (e.g., m/z 573, 591, and 595 in FIG. 3a).

To elucidate the composition of the C-terminal thermal decomposition products, accurate mass measurements of the ion at m/z 505.3 corresponding to the SEQ ID NO: 14 ⁻³²CKRM peptide thermal decomposition product (see FIG. 1a) and the ion at m/z 591.4 corresponding to the peptide SEQ ID NO: 13 ⁻³²CLLFK (see FIG. 3a) were performed. The results, listed in Table 1, yielded information about the most likely empirical formula of the N-terminus side of these peptides.

TABLE 1
Accurate mass measurements of the C-terminal
thermal decomposition (TD) products

| C-Terminal | | | Theor. | N-terminus |
|---|---|---|---|---|
| TD peptide | Measured | Calculated | Mass | empirical |
| product | M | formula | (Δ, ppm) | formula |
| ⁻³²CKRM | 504.28358 | C₂₀H₄₀O₅N₈[32]S | 504.28424 | C₂H₆N |
| | | | (0.01) | |
| ⁻³²CLLFK | 590.37854 | C₃₀H₅₀O₆N₆ | 590.37918 | C₂H₆N |
| | | | (0.03) | |

The empirical formulae derived from the accurate mass measurements were subtracted from the empirical formula of the section of the peptide assumed to be unmodified, as established by MS/MS measurements shown in FIG. 1. For example, the empirical formula for the section of the peptide SEQ ID NO: 13 CLLFK assumed to be unmodified (i.e., SEQ ID NO: 15 LLFK) is C₂₈H₄₄O₆N₅, while the accurate mass measurement yielded the empirical formula C₃₀H₅₀O₆N₆, leaving an empirical formula balance of C₂H₆N for the N-terminus of the peptide. Accurate mass measurements of two C-terminal thermal decomposition products from two different peptides (SEQ ID NO: 1PHCKRM and SEQ ID NO: 3 AWRGCLLFK) led to the same empirical formula and the same observed mass change. This empirical formula fits with the formation of either an alanine or β-alanine N-terminus amino acid for the C-terminal thermal decomposition product peptide.

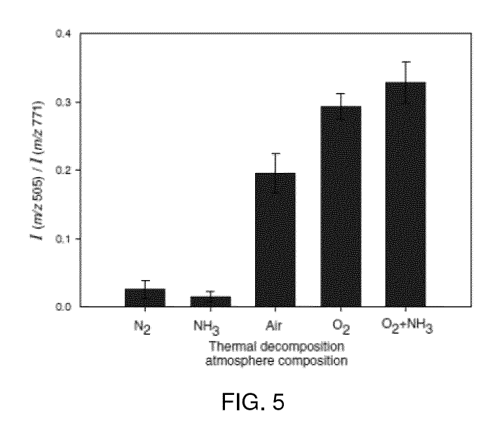

To gain insight into the mechanism for thermal decomposition cleavage at C, the role of molecular oxygen (O₂) in the possible initial oxidation of the C thiol side chain during the thermal decomposition process was investigated. When the thermal decomposition process was performed in an oxygen rich (˜100%) atmosphere it was found that cleavage product formation was enhanced by a factor of 1.5 when compared with the thermal decomposition process in an air atmosphere (FIG. 5). Furthermore, when the thermal decomposition was conducted in a N₂ rich (˜100%) atmosphere, no products resulting from the cleavage at C were detected, which suggest a direct role for oxygen in the C cleavage.

It is reasonable to assume that at the temperatures used in this study of 220-250° C. and in the presence of molecular oxygen, initial thiol side chain oxidation to either sulfonic and/or sulfinic acid occurs prior to the N—C_{α} bond cleavage. On the other hand, an elimination reaction with a net loss of H₂S is not likely to take place under these experimental conditions as they have been observed only at pyrolysis temperatures above 500° C. [1]. As a result, the mechanism for the thermally induced cleavage at the N-terminus of the cysteine side chain can be rationalized by a loss of the sulfur atom through an initial thiol group oxidation to sulfinic acid (R—SO₂H, see Scheme 3).

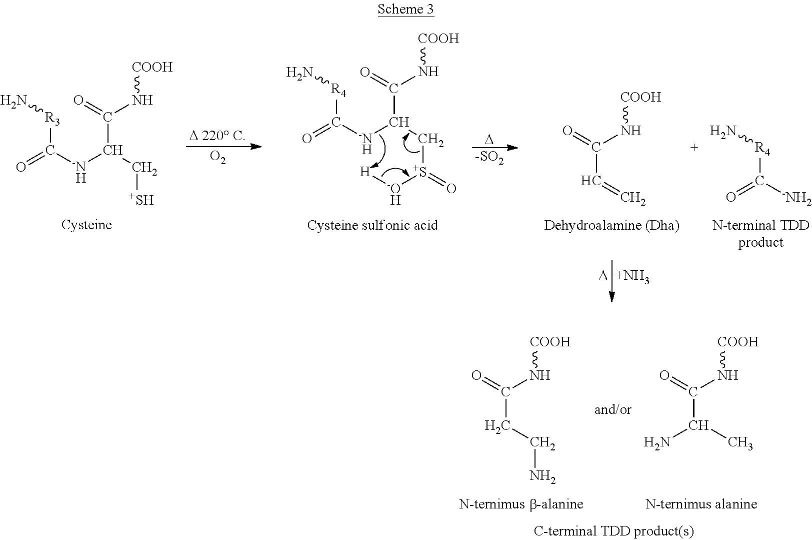

With the formation of a cysteine sulfinic acid, the production of an intermediate peptide fragment with a N-terminus dehydroalanine (Dha, ethylene moiety) is believed to take place via a N—C_{α} bond cleavage involving a concerted hydrogen abstraction and loss of SO₂. Direct evidence for the generation of SO₂ during the thermal decomposition of proteins can be found in early studies conducted by Kasarda and Black in 1968 [34], where a sealed glass tube containing a protein sample was heated and connected to an electron ionization (EI)-MS. In their study, they detected volatile thermal decomposition products from protein samples that included H₂O, NH₃, CO₂, H₂S, and SO₂, with the evolution of SO₂ starting at 220° C. and reaching a maximum intensity at a temperature of 265° C. (the origin of this SO₂ evolution was not known in that study). Under atmospheric conditions and high temperatures the intermediate Dha can undergo either an electrophilic addition (Markonikov orientation) or a free-radical addition (anti-Markonikov addition) with ammonia to yield either an alanine or β-alanine, respectively, as determined by accurate mass measurements (vide supra). Since the thermal decomposition reactions are being conducted in the condense phase, it is reasonable to assume that the proton source in Scheme 3 can be another acidic group from an adjacent peptide molecule (i.e., an intermolecular acid-base proton transfer). Also in Scheme 3, the thiol side chain in C can be oxidized to either sulfinic acid (R—SO₂H) or sulfonic acid (R—SO₃H). Experiments conducted with the peptide SEQ ID NO: 16 PHCₒₓKRM, where Cₒₓ is the C side chain oxidized to a sulfonic acid did not result in any of the expected nonvolatile products resulting from the cleavage at the N-terminus of C (data not shown).

The loss of ammonia during the thermal decomposition of peptides containing basic side chains is believed to be one of the sources of ammonia for the final formation of the C-terminus thermal decomposition product. Thermal decomposition experiments conducted in an atmosphere of approximately equimolar amounts of O₂ and NH₃ gasses (FIG. 5) resulted in higher levels of detected products than when performed in air. This last result concurs with the proposed formation of a Dha prior to addition of ammonia (electrophilic and/or free-radical) to form the C-terminus thermal decomposition product. Accordingly, it is expected that the addition of water to the intermediate Dha can also take place to form a corresponding C-terminal thermal decomposition product. A water addition to a Dha would yield a peptide with a hydroxyl group at the N-terminus instead of an amine group. This peptide resulting from the addition of water is expected to have a lower ionization efficiency when analyzed by ESI than the alanine/β-alanine products. Following this rationalization, the presence of the hydroxyl-substituted N-terminal product was confirmed by MS/MS of the ion at m/z 506, and the resulting tandem mass spectrum was compared with the tandem mass spectrum of the ion at m/z 505, corresponding to the alanine/β-alanine products (Figure S-3, Supplementary Material).

### Thermal Degradation/Digestion (TDD) Products of Intact Proteins

The TDD products of several intact proteins were also characterized in order to assess the method's potential utility as a rapid digestion step for intact proteins. The standard digestion method in bottom-up proteomics often utilizes the enzyme trypsin to induce hydrolysis at the C-terminus of arginine (R) and lysine (K), except when next to a proline residue. It is useful at this stage to compute the expected peptide length when the TDD method is used on several proteins. For a set of 30 E. coli proteins (in silico digestion,) the average number of amino acids in a peptide produced by the TDD method is 14 (±13) amino acids, while digestion with the enzyme trypsin is 9.6 (±9.2; 1 standard deviation). Moreover, median values for the peptide length produced by the TDD method is 10 amino acids, while for trypsin digestion is seven amino acids, illustrating that the median value is more representative of the central tendency of these distributions. The length of the resulting peptides can be explained by a 12% combined abundance of R and K in proteins, while the combined abundance of C and D in proteins is about 7%. Overall, longer peptides can be expected for the TDD method than for a trypsin digest. For example, the TDD method can potentially generate 41 peptides (out of 659 peptides) with amino acid lengths larger than 40 amino acids, while trypsin digestion of proteins yields only 10 peptides (out of 942 peptides) with 40 or more amino acids.

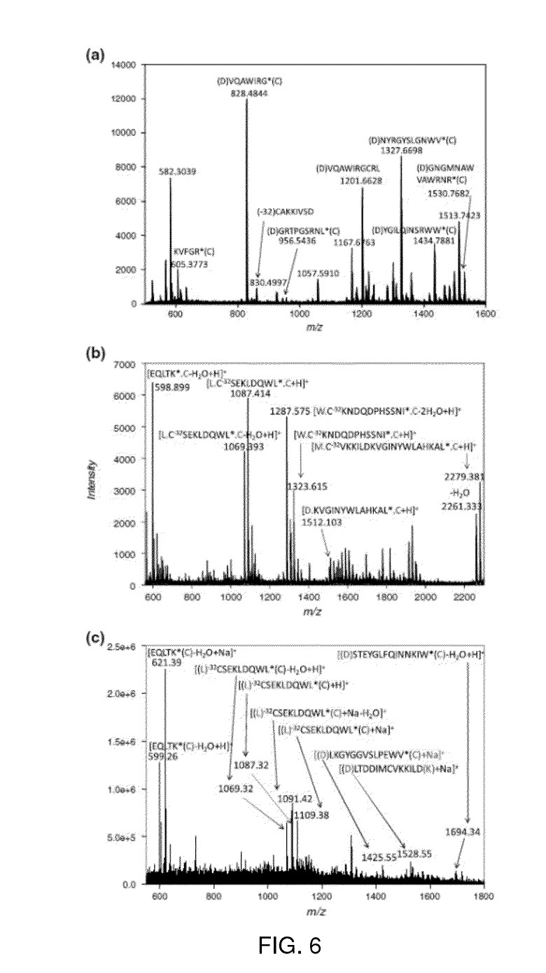

Mass spectra of the nonvolatile TDD products of two protein standards, lysozyme (chicken, egg white, 14.3 kDa) and α-lactalbumin (bovine, 14.2 kDa) were obtained and are shown in FIG. 6. The MALDI-mass spectra of the nonvolatile pyrolysis products of these proteins vary in terms of number of peptides detected and protein sequence coverage, reflecting both the sequence-specific nature of the TDD process and possible suppression ionization effects common in the MS analysis of peptide mixtures, as no LC step was performed in this study. For the protein lysozyme, the MALDI-mass spectrum in FIG. 6a features a wide range of peptide products stemming from both cleavages at the C-terminus of D and at the N-terminus of C. For example, the peptide detected at m/z 828.484 (SEQ ID NO: 17 VQAWIRG-NH₂, calculated MH⁺ monoisotopic mass 828.469 Da) was the result of a combined thermal cleavage at the C-terminus of D (D-119 in the sequence) and N-terminus of C (C-127 in the sequence), the latter leading to the amidation of the C-terminus G. Also, the peptide detected at m/z 1201.663 corresponds to the C-terminus end of the protein (cleavage at D-119) and shows a missed cleavage at C (C-127) [2]. This same peptide with two consecutive ammonia losses (two R groups in its sequence) is also detected at m/z 1167.676. Sequence confirmation of the ions at m/z 605.37, 828.48, 1327.67, and 1434.79 observed in FIG. 6a was performed by ESI-MS/MS. Overall, protein sequence coverage of 52% was obtained for lysozyme. Worth noting is the fact that there are several disulfide bonds within lysozyme and that these must have been broken during the TDD process in order to observe the peptides present in the mass spectrum in FIG. 6a. For example, disulfide bonds must be broken in order to generate the observed peptide fragments of sequences SEQ ID NO: 18 KVFGR*(C) (MH⁺ at m/z 605.378; asterisk indicates amidated C-terminus), SEQ ID NO: 19 (D)NYRGYSLGNWV*(C) (MH⁺ at m/z 1327.770), SEQ ID NO: 20 (D)YGILQINSRWW*(C) (MH⁺ at m/z 1434.788), SEQ ID NO: 21 (D)GRTPGSRNL*(C) (MH⁺ at m/z 956.544), SEQ ID NO: 22 (D)VQAWIRGCRL (MH⁺ at m/z 1201.663), and SEQ ID NO: 23 (D)VQAWIRG*(C) (MH⁺ at m/z 828.484). The detection of these peptides provides preliminary evidence that disulfide bonds are broken in lysozyme during the TDD process; however, further work is currently under way to confirm and elucidate this disulfide bond fragmentation mechanism.

The TDD products of the protein α-lactalbumin were analyzed by both MALDI-MS and ESI-MS (the same sample was split and analyzed by both techniques) and their mass spectra are shown in FIGS. 6b and c, respectively. The MALDI-mass spectrum in FIG. 6b shows signals of peptide products resulting from thermally induced cleavages at both D and C amino acids, in addition to peptides corresponding to dehydration products, most likely due to water loss from acidic groups like D and E. FIG. 6c shows the ESI-mass spectrum of the TDD products of the protein a-lactalbumin, where several ions observed in the MALDImass spectrum in FIG. 6b are also present, mainly ions at m/z 599.26, 1069.32, and 1087.32. In addition, several peptides were only detected by either MS analysis, illustrating the complementary nature between MALDI and ESI. Sequence confirmation of the ions at m/z 599, 1087, and 1693 was performed by ESI-MS/MS. With the combined results from the MALDI and ESI-MS measurements in FIGS. 6b and c, a sequence coverage of 64% is obtained for α-lactalbumin using the TDD method. Table 2 lists all the peptides detected from the TDD of α-lactalbumin by MALDI-MS and/or ESI-MS. It is expected that higher sequence coverages would be possible by incorporation of a LC step prior to ESI-MS.

TABLE 2
Peptides detected by either MALDI and/or ESI-MS from the TDD of the protein
α-lactalbumin (peptides cleaved at C have an amidated C-terminus)

| | | m/z | | |
|---|---|---|---|---|
| Peptide sequence | Ion | (nominal) | ESI | MALDI |
| SEQ ID NO: 24 EQLTK(C) | [M-H₂O + H]⁺ | 599 | X | X |
| SEQ ID NO: 24 EQLTK(C) | [M-H₂O + Na]⁺ | 621 | X | |
| SEQ ID NO: 25 (L)⁻³²CSEKLDQWL(C) | [M + H]⁺ | 1087 | X | X |
| SEQ ID NO: 25 (L)⁻³²CSEKLDQWL(C) | [M-H₂O + H]⁺ | 1069 | X | X |
| SEQ ID NO: 25 (L)⁻³²CSEKLDQWL(C) | [M-H₂O + Na]⁺ | 1091 | X | X |
| SEQ ID NO: 26 (W)⁻³²CKNDQDPHSSNI(C) | [M + H]⁺ | 1324 | | X |
| SEQ ID NO: 26 (W)⁻³²CKNDQDPHSSNI(C) | [M-2H₂O + H]⁺ | 1288 | | X |
| SEQ ID NO: 27 (D)LKGYGGVSLPEWV(C) | [M + Na]⁺ | 1426 | X | |
| SEQ ID NO: 28 (D)KVGINYWLAHKAL(C) | [M + H]⁺ | 1512 | | X |
| SEQ ID NO: 29 (D)LTDDIMCVKKILD(K) | [M + Na]⁺ | 1529 | X | |
| SEQ ID NO: 30 (D)STEYGLFQINNKIW(C) | [M-H₂O + H]⁺ | 1694 | X | |
| SEQ ID NO: 31 (M)⁻³²CVKKILDKVGINYWLAHKAL(C) | [M + H]⁺ | 2279 | | X |
| SEQ ID NO: 31 (M)⁻³²CVKKILDKVGINYWLAHKAL(C) | [M-H₂O + H]⁺ | 2261 | | X |

### Conclusions

Conclusive evidence was presented for the thermally induced site-specific cleavage at the N-terminus of the amino acid C in peptides and proteins. Also demonstrated in this work was the simultaneous cleavage at D and C under the same experimental conditions in protein standards. Mass spectrometry studies combining a peptide containing a ¹⁵N stable isotope, peptides with oxidized C, MS/MS, and accurate mass measurements of the TDD products revealed a cleavage at the N-terminus of C. Studies showed that this thermally induced cleavage at C most likely involves an initial thiol group oxidation to sulfinic acid, and thus requires the presence of molecular oxygen (i.e., performed under atmospheric conditions). The cleavage at the N-terminus of C proceeds by a concerted N—C_{α} bond cleavage and loss of SO₂, forming an intermediate Dha at the N-terminus of the C-terminal TDD product. Addition of ammonia (or water) to this Dha moiety is believed to be the final step in the formation of either an alanine and/or β-alanine C-terminal TDD product. Tandem MS measurements of the C- and N-terminal TDD products of peptide and protein standards showed preserved information of the direct amino acid sequence, pointing to the chemical site-specificity of the method and its compatibility with MS detection. Evidence from the analysis of proteins with TDD presented in this study also showed that disulfide bonds are cleaved during this process, and further work is currently under way to elucidate the disulfide bond fragmentation mechanism by TDD. Given the TDD method speed (10 s), reagentless nature, site-specificity and the moderate length of the peptides produced, current work in our laboratory is exploring the utility of the TDD method as part of a proteomic workflow for rapid protein identification.

### EXAMPLE 2

### Experimental

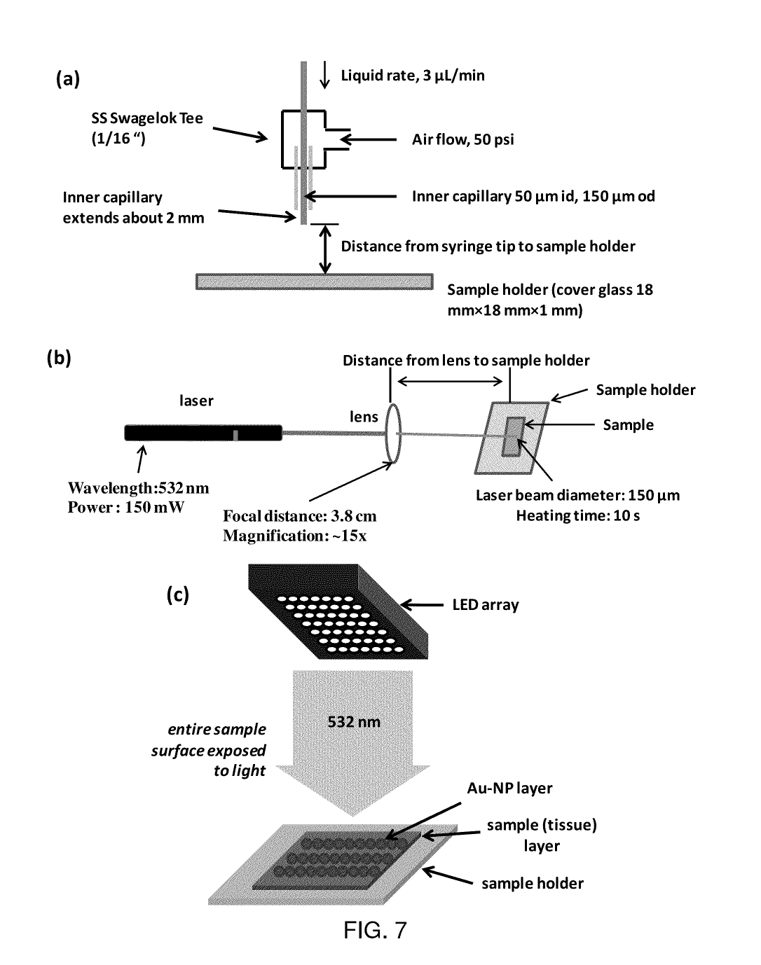

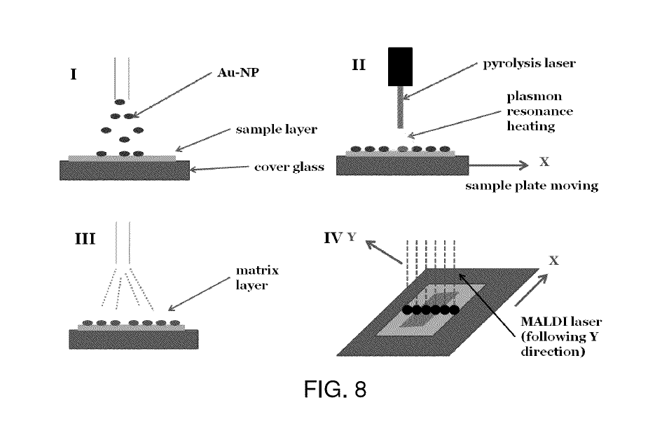

The experimental apparatus is illustrated in FIGS. 7 and 8. FIG. 7(a) illustrates the detailed diagram of an oscillating capillary nebulizer (OCN) used to spray samples with Au-NP and MALDI matrix solutions. Any other device can be used to spread an uniform film of the Au NP. FIG. 7(b) shows the setup of Photo-TDD using a laser. Laser used in the photo-TDD experiments was 150 mW of power and 532 nm of wavelength, and this laser was focused by lens with focal distance 38 mm and magnification 15 times. FIG. 7(c) shows the setup for Photo-TDD using a LED array for exposing the entire sample area to light. FIG. 8 shows the stepwise processes of photo-TDD experiment using a laser and Au-NP's in conjunction with MALDI-MS detection.

After being sprayed with Au NP's by the OCN setup, the sample was exposed by this focused laser in an appointed position, and the sample holder was moved at a constant rate, effectively scanning the laser across the sample. By changing the sample moving rate, the heating time of the photo-TDD process onto the sample was altered. Meanwhile, by changing the distance from lens to the sample holder, the laser beam was focused to different light intensities, and thus, temperatures.

As shown in FIG. 8(I), the sample solution (a peptide or protein) was sprayed via the OCN setup, followed by the Au-NP solution deposited in the same place. In FIG. 8(II), a focused laser beam was scanned across this sample to induce Photo-TDD. In FIG. 8(III), the MALDI matrix was spray-deposited on top of the laser heated area. In FIG. 8(IV), detection of the photo-TDD products by MALDI-TOF-MS was performed by moving the MALDI laser across the path of the Photo-TDD laser beam in order to obtain a spatial cross section of the products.

### Results and Discussion

The utility of Au-NP surface Plasmon resonance assisted photo-TDD for performing site-specific digestions on tissue was investigated with several types of peptides, which included peptides with either aspartic acid (D), cysteine (C), or with both C and D or two D's.

Site specific cleavages at the aspartic acid (D) and cysteine (C) could be induced by heating the sample to a temperature of 220° C.-250° C. for 10 s under atmospheric pressure condition.^{2,37} The implementation of photo-TDD removes the need to extract product peptides from tissues and/or vials as the digestion and detection is performed in situ, that is, on-tissue. In our previous published articles, biomolecules, peptides and proteins, were heated in a test tube furnace, TDD products extracted with solvent, followed by ESI-MS, MS/MS and MALDI-TOF-MS analysis to characterize and identify produced TDD fragments.^{2, 26-38} In these Photo-TDD examples, several standard peptides undergo photo-TDD and products analyzed in situ by MALDI-TOF-MS.

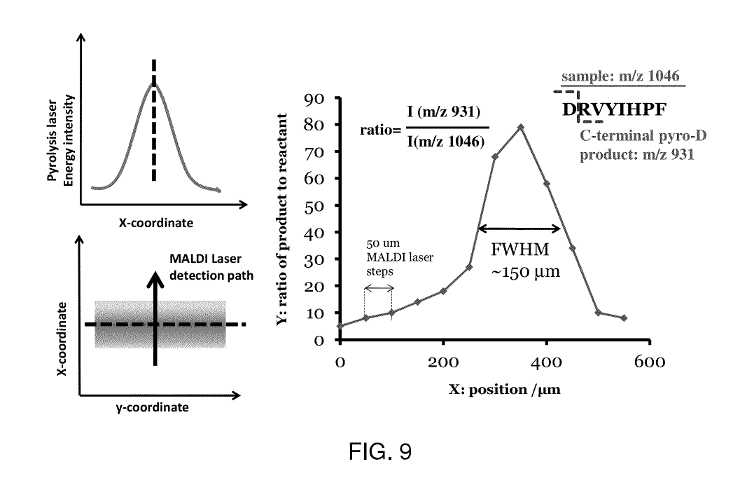

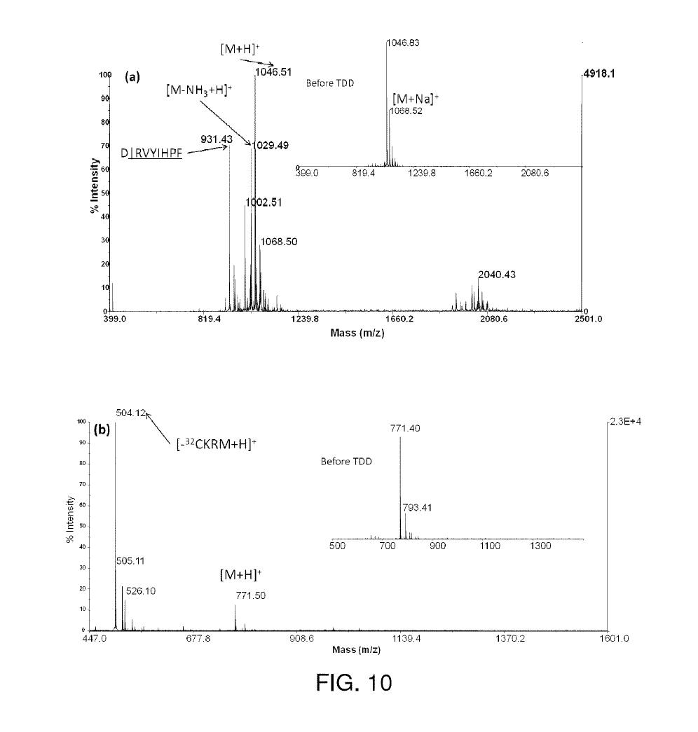

FIG. 9 shows the results of the experiment outlined in FIG. 8, yielding a spatial resolution of 150 micro-meters for an un-optimized Photo-TDD system. FIG. 10(a) illustrates the mass spectra of the peptide Angiotensin II before (inset) and after photo-TDD. The calculated Au-NP surface concentration of this experiment is 6.34×10⁸ NPs/mm². The sample is shined by the focused laser in a position 5.5 cm away from the lens, and the heating time is 10 s. The mass spectrum after photo-TDD shows the TDD product at m/z 931, resulting from the cleavage at the C-terminus of the amino acid D. Also observed are the deamination and dehydration products at m/z 1029 and m/z 1028, respectively, which were in agreement with results previously reported using the test tube furnace pyrolyzer. FIG. 10(b) shows the mass spectra of the peptide antioxidant peptide-A before and after photo-TDD. The calculated Au-NP surface concentration of this experiment is 1.26×10⁹ NPs/mm². The sample is shined by the focused laser in a position 5.5 cm away from the lens, and the heating time is 10 s. In the mass spectrum of after photo-TDD, fragments from the cleavage at N terminal of cysteine were detected at m/z 505 and the peak m/z 504.

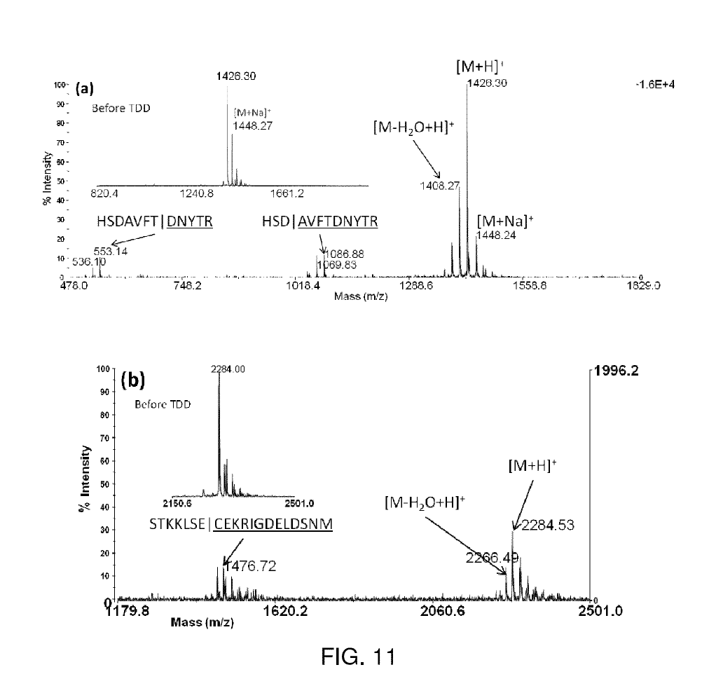

FIG. 11(a) shows results the photo-TDD experiment for another peptide, VIP (1-12), which contains two aspartic acid (D) residues. The calculated Au-NP surface concentration of this experiment is 6.34×10⁸ NPs/mm². The sample is shined by the focused laser in a position 5.5 cm away from the lens, and the heating time is 10 s. In the mass spectrum of the Photo-TDD products the expected products were observed at m/z 553 and 1086. Furthermore, deamination product at m/z 1069 from ion 1086 and dehydration product of primary ion at m/z 1408 were also observed. In FIG. 11(b), another peptide Bax, BH3, containing one cysteine residue and two aspartic acid residues was analyzed by MALDI-MS before and after photo-TDD. The calculated Au-NP surface concentration of this experiment is 6.34×10⁸ NPs/mm². The sample is exposed to the focused laser in a position 5.5 cm away from the lens, and the heating time is 10 s. In the mass spectrum of the Photo-TDD products, a signal corresponding to the cleavage at the N-terminal of cysteine residue was observed at m/z 1476.

The above results demonstrated that the photo-TDD, like the furnace pyrolyzer TDD method, can cleave peptides in specific sites at cysteine and aspartic acid in 10 s.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

1. Moldoveanu S. C.: Pyrolysis of Organic Molecules with Applications to Health and Environmental Issues Vol. 28; Elsevier: Amsterdam, (2010)
2. Zhang S., Basile, F.: Site-specific pyrolysis-induced cleavage at aspartic acid residue in peptides and proteins. J. Proteome Res. 6, 1700-1704 (2007)
3. Hacaloglu J., Yalcin, T., Oenal, A. M.: Pyrolysis studies to investigate effects of polymerization techniques on structure and thermal behavior of poly(1,2-epoxy-4-epoxyethylcyclohexanes). J. Macro. Sci. Pure Appl. Chem. A32, 1167-1181 (1995)
4. Blazso M.: Recent trends in analytical and applied pyrolysis of polymers. J. Anal. Appl. Pyrolysis 39, 1-25 (1997)
5. Posthumus M. A., Boerboom, A. J. H., Meuzelaar, H. L. C.: Analysis of biopolymers by Curie-point pyrolysis in direct combination with low-voltage electron impact ionization mass spectrometry. Advances Mass Spectrom. 6, 397-402 (1974)
6. Ballistreri A., Giuffrida, M., Maravigna, P., Montaudo, G.: Direct mass spectrometry of polymers. XII. Thermal fragmentation processes in poly (a-amino acids). J. Polym. Sci. Part A Polym. Chem. 23, 1145-1161 (1985)
7. Anhalt J. P., Fenselau, C.: Identification of bacteria using mass spectrometry. Anal. Chem. 47, 219-225 (1975)
8. Beverly M. B., Basile, F., Voorhees, K. J., Hadfield, T. L.: A rapid approach for the detection of di-picolinic acid in bacterial spores using pyrolysis/mass spectrometry. Rapid Commun. Mass Spectrom. 10, 455-458 (1996)
9. Wieten G., Meuzelaar, H. L. C., Haverkamp, J.: Analytical pyrolysis in clinical and pharmaceutical microbiology. In Odham, G.; Larsson, L.; Maardh, P.-A., (eds.) Gas Chromatogr./Mass Spectrom. Appl. Microbiol, pp 335-380. Plenum, (1984)
10. Dworzanski J. P., Tripathi, A., Snyder, A. P., Maswdeh, W. M., Wick, C. H.: Novel biomarkers for gram-type differentiation of bacteria by pyrolysis-gas chromatography-mass spectrometry. J. Anal. Appl. Pyrolysis 73, 29-38 (2005)
11. Voorhees K. J., Durfee, S. L., Updegraff, D. M.: Identification of diverse bacteria grown under diverse conditions using pyrolysis-mass spectrometry. J. Microbiol. Methods 8, 315-325 (1988)
12. DeLuca S., Sarver, E. W., Harrington, P. d. B., Voorhees, K. J.: Direct analysis of bacterial fatty acids by Curie-point pyrolysis tandem mass spectrometry. Anal. Chem. 62, 1465-1472 (1990)
13. Basile F., Beverly, M. B., Abbas-Hawks, C., Mowry, C. D., Voorhees, K. J., Hadfield, T. L.: Direct mass spectrometric analysis of in situ thermally hydrolyzed and methylated lipids from whole bacterial cells. Anal. Chem. 70, 1555-1562 (1998)
14. Fenn J. B., Mann, M., Meng, C. K., Wong, S. F., Whitehouse, C. M.: Electrospray ionization for mass spectrometry of large biomolecules. Science 246, 64-71. (1989)
15. Fenn J. B., Mann, M., Meng, C. K., Wong, S. F., Whitehouse, C. M.: Electrospray ionization—principles and practice. Mass Spectrom. Rev. 9, 37-70 (1990)
16. Kebarle P., Verkerk, U.: On the mechanism of electrospray ionization mass spectrometry (ESIMS). In: Cole, R. B., (ed.) Electrospray and MALDI Mass Spectrometry, 2nd ed., pp 3-48. John Wiley and Sons, Inc. (2010)
17. Karas M., Bachmann, D., Hillenkamp, F.: Influence of the wavelength in high-irradiance ultraviolet laser desorption mass spectrometry of organic molecules. Anal. Chem. 57, 2935-2939 (1985)
18. Karas M., Hillenkamp, F.: Laser desorption ionization of proteins with molecular masses exceeding 10,000 daltons. Anal. Chem. 60, 2299-2301 (1988)
19. Karas M., Bahr, U., Ingendoh, A., Hillenkamp, F.: Laser-desorption mass spectrometry of 100,000-250,000 Da proteins. Angew. Chem. 101, 805-806 (1989)
20. Knochenmuss R.: MALDI ionization mechanisms: an overview. In Electrospray and MALDI Mass Spectrometry 2nd ed., pp 149-183. John Wiley and Sons, Inc. (2010)
21. Lattimer R. P., Polce, M. J., Wesdemiotis, C.: MALDI-MS analysis of pyrolysis products from a segmented polyurethane. J. Anal. Appl. Pyrolysis 48, 1-15 (1998)
22. Lattimer R. P.: Mass spectral analysis of low-temperature pyrolysis products from poly(ethylene glycol). J. Anal. Appl. Pyrolysis 56, 61-78 (2000)
23. Meetani M. A., Basile, F., Voorhees, K. J.: Investigation of pyrolysis residues of poly(amino acids) using matrix assisted laser desorption ionization-time of flight-mass spectrometry. J. Anal. Appl. Pyrolysis 68/69, 101-113 (2003)
24. Meetani M. A., Zahid, O. K., Michael Colon, J. M.: Investigation of the pyrolysis products of methionine-enkephalin-Arg-Gly-Leu using liquid chromatography tandem mass spectrometry. J. Mass Spectrom. 45, 1320-1331 (2010)
25. Zhang S., Shin, Y.-S., Mayer, R., Basile, F.: On-probe pyrolysis desorption electrospray ionization (DESI) mass spectrometry for the analysis of nonvolatile pyrolysis products. J. Anal. Appl. Pyrolysis 80, 353-359 (2007)
26. Chen X., Yu, L., Steill, J. D., Oomens, J., Polfer, N. C.: Effect of peptide fragment size on the propensity of cyclization in collisioninduced dissociation: Oligoglycine b2-b8. J. Am. Chem. Soc. 131, 18272-18282 (2009)
27. Molesworth S., Osburn, S., Van Stipdonk, M.: Influence of size on apparent scrambling of sequence during CID of b-type ions. J. Am. Soc. Mass Spectrom. 20, 2174-2181 (2009)
28. Chen, X., Steill, J. D., Oomens, J., Polfer, N. C.: Oxazolone versus macrocycle structures for Leu-enkephalin b2-b4: insights from infrared multiple-photon dissociation spectroscopy and gas-phase hydrogen/deuterium exchange. J. Am. Soc. Mass Spectrom. 21, 1313-1321 (2010)
29. Gucinski, A. C., Somogyi, A., Chamot-Rooke, J., Wysocki, V. H.: Separation and identification of structural isomers by quadrupole collision-induced dissociation-hydrogen/deuterium exchange-infraredmultiphoton dissociation (QCID-HDX-IRMPD). J. Am. Soc. Mass Spectrom. 21, 1329-1338 (2010)
30. Bythell B. J., Knapp-Mohammady, M., Paizs, B., Harrison, A. G.: Effect of the His Residue on the Cyclization of b Ions. J. Am. Soc. Mass Spectrom. 21, 1352-1363 (2010)
31. ABRF Delta Mass: A Database of Protein Post Translational Modifications. http://www.abrf.org/index.cfm/dm.home?AvgMass=all; accessed on November 10, (2010)
32. Paizs B., Suhai, S.: Fragmentation pathways of protonated peptides. Mass Spectrom. Rev. 24, 508-548 (2005)
33. Ballard K. D., Gaskell, S. J.: Dehydration of peptide [M+H]+ ions in the gas phase. J. Am. Soc. Mass Spectrom. 4, 477-481 (1993)
34. Kasarda D. D., Black D. R.: Thermal degradation of proteins studied by mass spectrometry. Biopolymers 6, 1001-1004 (1968)
35. Henzel, W. J.; Billeci, T. M.; Stults, J. T., Wong, S. C., Grimley, C., Watanabe, C.: Identifying proteins from two-dimensional gels by molecular mass searching of peptide fragments in protein sequence databases. Proc. Natl. Acad. Sci. U.S.A., 1;90(11), 5011-5 (1993)
36. Huaser, N. J.; Basile, F. On-line Microwave D-Cleavage LC-ESI-MS/MS of Intact Proteins: Site-Specific Cleavages at Aspartic Acid Residues and Disulfide Bonds. Journal of Proteome Research, 2008. 7: 1012-1026 (2008)
37. Basile, F.; Shang, S.; Kandar, S.; Lu, L. Mass Spectrometry Characterization of the Thermal Decomposition/Digestion (TDD) at Cysteine in Peptides and Proteins in the Condensed Phase, J. Am. Soc. Mass Spectrom., 22, 1926-1940 (2011)
38. Basile, F.; Kassalainen, G. E.; Ratanathanawongs Williams, S. K. A Simple Interface for Direct and Continuous Sample-Matrix Deposition onto a MALDI Probe for Polymer Analysis by Thermal Field Flow Fractionation and Off-line MALDI-MS, Anal. Chem., 77, 3008-3012 (2005)

1. A method for decomposing or digesting peptides and proteins, comprising the steps of:
(a) applying to a sample containing peptides or proteins a responsive composition that heats upon exposure to light;
(b) shining a light source on at least a portion of the sample and responsive composition to heat the portion to a temperature and for between 1 and 30 seconds sufficient to cause decomposition or digestion of the peptides or proteins at the C-terminal of aspartic acid and at the N-terminal of cysteine.

2. A method of claim 1, wherein the heat is created by surface plasmon resonance.

3. A method of claim 1, wherein the responsive composition comprises gold nanoparticles.

4. A method of claim 3, wherein the gold nanoparticles are in the range of between 10 and 200 nm and preferably between 30 and 90 nm.

5. A method of claim 3, wherein the light source includes green light.

6. A method of claim 4 wherein the light is light with a wavelength centered at 532 nm.

7. A method of claim 5, wherein the light is selected from the group consisting of laser light and light from an LED.

8. A method of claim 1, wherein the temperature is between 220 and 250° C.

9. (canceled)

10. (canceled)

11. Apparatus for decomposing or digesting peptides and proteins, comprising:
(a) a sample containing peptides or proteins;
(b) a responsive composition that heats upon exposure to light applied to a portion of the sample;
(c) a light source that is shined on at least part of the portion of the sample and responsive composition to heat the part to a temperature and for between 1 and 30 seconds sufficient to cause decomposition or digestion of the peptides or proteins in the part at the C-terminal of aspartic acid and at the N-terminal of cysteine; and
(d) apparatus for detecting the decomposed or digested peptides or proteins.

12. Apparatus of claim 11, wherein the heat is created by surface plasmon resonance.

13. Apparatus of claim 11, wherein the responsive composition comprises gold nanoparticles.

14. Apparatus of claim 13, wherein the gold nanoparticles are in the range of between 10 and 200 nm.

15. Apparatus of claim 13, wherein the light source shines green light.

16. Apparatus of claim 14 wherein the light is light with a wavelength centered at 532 nm.

17. Apparatus of claim 15, wherein the light is selected from the group consisting of laser light and light from an LED.

18. Apparatus of claim 11, wherein the temperature is between 220 and 250° C.

19. (canceled)

20. (canceled)


SEQUENCE LISTING (31 sequences)

SEQ ID NO: 1 - protein, 7 aa, Artificial sequence
  Experimental peptide
PHCLKRM

SEQ ID NO: 2 - protein, 16 aa, Artificial sequence
  Experimental peptide
AGCLKNFFWLKTFTSC

SEQ ID NO: 3 - protein, 10 aa, Artificial sequence
  Experimental peptide
AWRGCLLFLK

SEQ ID NO: 4 - protein, 4 aa, Artificial sequence
  Experimental peptide
AWRG

SEQ ID NO: 5 - protein, 4 aa, Artificial sequence
  Experimental peptide
MRFA

SEQ ID NO: 6 - protein, 4 aa, Artificial sequence
  Experimental peptide
FGFG

SEQ ID NO: 7 - protein, 5 aa, Artificial sequence
  Experimental peptide
YGGFM

SEQ ID NO: 8 - protein, 7 aa, Artificial sequence
  Experimental peptide
YGGFLLK

SEQ ID NO: 9 - protein, 5 aa, Artificial sequence
  Experimental peptide
YGGFL

SEQ ID NO: 10 - protein, 5 aa, Artificial sequence
  Experimental peptide
CLKRM

SEQ ID NO: 11 - protein, 5 aa, Artificial sequence
  Experimental peptide
MCLKR

SEQ ID NO: 12 - protein, 11 aa, Artificial sequence
  Experimental peptide
CNFFWLKTFTS

SEQ ID NO: 13 - protein, 6 aa, Artificial sequence
  Experimental peptide
CLLFLK

SEQ ID NO: 14 - protein, 5 aa, Artificial sequence
  Experimental peptide
CLKRM

SEQ ID NO: 15 - protein, 5 aa, Artificial sequence
  Experimental peptide
LLFLK

SEQ ID NO: 16 - protein, 7 aa, Artificial sequence
  Experimental peptide
PHCLKRM

SEQ ID NO: 17 - protein, 7 aa, Artificial sequence
  Experimental peptide
VQAWIRG

SEQ ID NO: 18 - protein, 7 aa, Artificial sequence
  Experimental peptide
LKVFGRC

SEQ ID NO: 19 - protein, 13 aa, Artificial sequence
  Experimental peptide
DNYRGYSLGNWVC

SEQ ID NO: 20 - protein, 13 aa, Artificial sequence
  Experimental peptide
DYGILQINSRWWC

SEQ ID NO: 21 - protein, 11 aa, Artificial sequence
  Experimental peptide
DGRTPGSRNLC

SEQ ID NO: 22 - protein, 11 aa, Artificial sequence
  Experimental peptide
DVQAWIRGCRL

SEQ ID NO: 23 - protein, 9 aa, Artificial sequence
  Experimental peptide
DVQAWIRGC

SEQ ID NO: 24 - protein, 7 aa, Artificial sequence
  Experimental peptide
EQLTLKC

SEQ ID NO: 25 - protein, 12 aa, Artificial sequence
  Experimental peptide
LCSELKLDQWLC

SEQ ID NO: 26 - protein, 15 aa, Artificial sequence
  Experimental peptide
WCLKNDQDPHSSNIC

SEQ ID NO: 27 - protein, 16 aa, Artificial sequence
  Experimental peptide
DLLKGYGGVSLPEWVC

SEQ ID NO: 28 - protein, 17 aa, Artificial sequence
  Experimental peptide
DLKVGINYWLAHLKALC

SEQ ID NO: 29 - protein, 18 aa, Artificial sequence
  Experimental peptide
DLTDDIMCVLKLKILDLK

SEQ ID NO: 30 - protein, 17 aa, Artificial sequence
  Experimental peptide
DSTEYGLFQINNLKIWC

SEQ ID NO: 31 - protein, 26 aa, Artificial sequence
  Experimental peptide
MCVLKLKILDLKVGINYWLAHLKALC